(19) United States
(12) Patent Application Publication
(10) Pub. No.: US 2010/0305613 A1
(43) Pub. Date: Dec. 2, 2010

(54) Headless Polyaxial Screw System

(75) Inventors: Mahmoud F. Abdelgany, Rockaway, NJ (US); Mohamed Ahmed Hafez Ramadan, Cairo (EG)

Correspondence Address: Rahman LLC;Mohammad Rahman, 10025 Governor Warfield Parkway, Suite 110, Columbia, MD, 21044, US

(73) Assignee: Custom Spine, Inc., Parsippany, NJ (US)

(21) Appl. No.: 12/474,789

(22) Filed: May 29, 2009

(51) Int. Cl. A61B 17/70; A61B 17/86; A61B 17/84

(52) U.S. Cl. 606/264; 606/305; 606/330

(57) ABSTRACT

A headless polyaxial screw system comprises a longitudinal member comprising an outwardly protruding and expandable round bulbous body, a fixation component directly connected to the bulbous body, where the fixation component receives the bulbous body, and a pin engaged within the longitudinal member via a first channel bored through the longitudinal member and contacting the bulbous body causing the bulbous body to outwardly expand.

### BACKGROUND

1. Technical Field

The embodiments herein generally relate to medical devices and assemblies, and more particularly to an orthopedic surgical implant assembly used in the field of surgical lumbar, thoracic, and cervical spine treatment.

2. Description of the Related Art

Surgical procedures treating spinal injuries are one of the most complex and challenging surgeries for both the patient and the surgeon. When there are various deformities, trauma, or fractures of the vertebra, surgeons may attempt to “fuse” them together by attaching screw-like devices into the pedicles of the spine and thereby connecting several vertebrae (typically two or more) using a semi-rigid rod. However, due to the complexity of the human anatomy, most surgeons must bend the rod (causing notches thereby reducing fatigue resistance) before placing them into two or more non-aligned pedicle screws in order to properly stabilize the pedicle screw assembly within the patient's body.

Depending on the purpose of the spine surgery, indications, and patient size, surgeons must pre-operatively choose between different spinal systems with differing rod sizes pre-operatively sometimes causing delays in surgery while waiting for more adequate systems to be sterilized. Some surgeons prefer monoaxial screws for rigidity, while some sacrifice rigidity for surgical flexibility in screw placement. Therefore, a system is needed to accommodate both theories. For example, during scoliosis surgery conventional polyaxial systems typically cannot lock into a desired position to persuade the spinal column into desired correction before final construct assembly.

Most conventional top loading polyaxial spine screws do not do enough to address cantilever failure of the assembly components. Additionally, conventional systems require several different components to be manipulated and assembled by the surgeon during a surgical procedure. Reducing the number of components in a screw assembly that are manipulated and assembled would simplify operating room logistics; the steps performed by the surgeon during the surgical procedure; and, ultimately, improve patient recovery time. Thus, there remains a need for a new and improved pedicle screw assembly capable of overcoming the limitations of the conventional designs thereby providing the surgeon with improved intra-operative flexibility and the patient with an improved prognosis for better and complete rehabilitation.

### SUMMARY

In view of the foregoing, an embodiment herein provides an assembly comprising: a longitudinal member comprising an outwardly protruding and expandable round bulbous body; a fixation component directly connected to the bulbous body, wherein the fixation component receives the bulbous body; and a pin engaged within the longitudinal member via a first channel bored through the longitudinal member and contacting the bulbous body causing the bulbous body to outwardly expand.

The assembly may further provide a fixation component that comprises a concave socket that receives the bulbous body of the longitudinal member. The fixation component may also comprise a bone screw. Additionally, the fixation component may comprise a hook.

The assembly may also provide a longitudinal member that comprises a substantially planar lower surface, wherein the bulbous body extends from the lower surface of the longitudinal member, and wherein the concave socket cups the expandable bulbous body. The assembly may also include a set screw engaged with the longitudinal member via a second channel bored through the longitudinal member. Furthermore, the assembly may provide a pin that engages with the fixation component. In addition, each of the first channel and second channel may be etched with threads.

Moreover, the bulbous body of the longitudinal member may comprise a plurality of slots separating a plurality of bendable flanges of the bulbous body.

Also provided is a system comprising a first assembly and a second assembly, wherein the first assembly comprises: a first longitudinal member comprising: a first elongated body outwardly extending from at least one longitudinal end of the first longitudinal member; and an outwardly protruding and expandable round bulbous body extending from a lower portion of the first longitudinal member, wherein the lower portion of the first longitudinal member is positioned substantially transverse to the at least one longitudinal end of the first longitudinal member; a fixation component directly connected to the bulbous body, wherein the fixation component receives the bulbous body; and a pin engaged within the longitudinal member via a first channel bored through the first longitudinal member and contacting the bulbous body causing the bulbous body to outwardly expand.

Such a system may also provide the first longitudinal member further comprising a first elongation channel bored therein, and the second assembly comprises a second longitudinal member comprising: a second elongated body outwardly extending from at least one longitudinal end of the second longitudinal member; and a second longitudinal member comprising a second elongation channel bored therein. Furthermore, the system may provide the first elongation channel that accepts the second elongated body, and the second elongation channel accepts the first elongated body.

The system may also provide any of the first longitudinal member of the first assembly and the second longitudinal member of the second assembly that further comprises a hole that receives a set screw. The system may further provide the set screw of the first assembly engages the second elongated body. In addition, the system may provide the set screw of the second assembly that engages the first elongated body.

An assembly is also provided that comprising a longitudinal member comprising at least two outwardly protruding and expandable round bulbous bodies; at least two fixation components directly connected to the bulbous bodies; and at least two pins engaged within the longitudinal member via channels bored through the longitudinal member, wherein each pin is set within each channel and contacts a bulbous body causing the bulbous body to outwardly expand to engage a fixation component.

Such an assembly may also provide each of the bulbous bodies that are angled with respect to each other. Furthermore, the assembly may provide the longitudinal member comprises at least one of an elongated body and an elongation channel.

Moreover, a method of engaging a pedicle fixation assembly to a vertebral body is provided, the method comprises attaching at least one bone fixation component to the vertebral body, where at least one bone fixation component comprises an open concave socket; directly attaching a longitudinal member to the bone fixation component, where the longitudinal member comprises an outwardly protruding and expandable round bulbous body that fits into the open concave socket; and inserting a pin through a first channel bored through the longitudinal member to contact the bulbous body causing the bulbous body to outwardly expand into the concave socket.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 illustrates a perspective view of a screw assembly according to an embodiment described herein;

FIG. 2 illustrates a perspective view of a longitudinal member according to an embodiment described herein;

FIG. 3 illustrates a cross-sectional view of a longitudinal member according to an embodiment described herein;

FIG. 4(A) illustrates a bottom view of the bulbous end of the longitudinal member of FIGS. 1 through 3 according to an embodiment described herein;

FIG. 4(B) illustrates a longitudinal cross-sectional view of a longitudinal member, according to an embodiment described herein;

FIG. 5(A) illustrates a perspective view of a securing pin according to an embodiment described herein;

FIG. 5(B) illustrates a perspective view of a set screw according to an embodiment described herein;

FIG. 5(C) illustrates a perspective view of a load-bearing component according to an embodiment herein;

FIG. 6 illustrates a perspective view of two adjoining screw assemblies according to an embodiment described herein;

FIG. 7 illustrates a perspective view of two adjoining screw assemblies secured to vertebrae according to an embodiment described herein;

FIG. 8 illustrates a perspective view of an alternative embodiment of a headless screw system according to an embodiment described herein;

FIG. 9 illustrates a illustrates a perspective view of a screw assembly according to yet another embodiment described herein;

FIGS. 10(A) and 10(B) illustrates perspective views of two adjoining screw assemblies secured to vertebrae according to yet another embodiment described herein; and

FIG. 11 is a flow diagram illustrating a preferred method according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

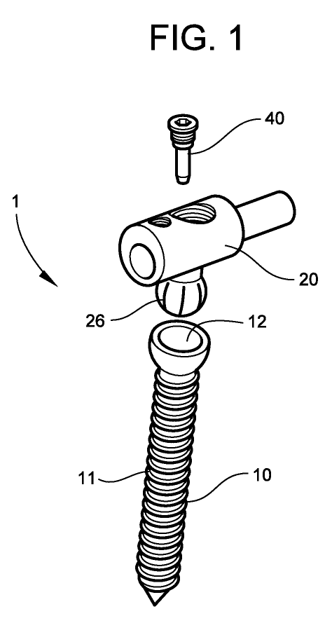

As mentioned, there remains a need for a new and improved pedicle screw assembly capable of overcoming the limitations of the conventional designs thereby providing the surgeon with improved intra-operative flexibility and the patient with an improved prognosis for better and complete rehabilitation. The embodiments herein address this need by providing an improved headless polyaxial screw device with fewer components than conventional systems and method of assembly capable of simplifying a surgical procedure using such an improved headless polyaxial screw device. Referring now to the drawings and, more particularly to FIGS. 1 through 11, where similar reference characters denote corresponding features consistently throughout the figures, there are shown preferred embodiments.

FIG. 1 illustrates a first embodiment of a headless polyaxial screw system 1. The screw assembly 1 comprises a fixation component 10 (e.g., a bone screw or bone anchor or hook, etc.), longitudinal member 20 (e.g., a spinal rod or plate) and securing pin 40. Fixation component 10 is shown having a threaded end 11 for engaging a bone (not shown in FIG. 1) and a concave female socket 12 for engaging and receiving the bulbous body 26 of longitudinal member 20 (as described in further detail below). Longitudinal member 20 is shown in just one of many possible configurations available for a longitudinal member, several more are discussed below. Accordingly, those skilled in the art, however, would recognize that other longitudinal member configurations are possible including square, rectangular, triangular, circular cross sections, and the embodiments described below do not limit alternative embodiments of the longitudinal member 20, specifically, or the headless polyaxial screw system, in general. During the manufacturing process, the screw assembly 1 may be prepared for transport by securing longitudinal member 20 to fixation component 10 via the securing pin 40 and subjecting screw assembly 1 to ultra sonic cleaning. In so doing, any impurities are removed from screw assembly 1 and subsequently may be shipped in this manufactured format.

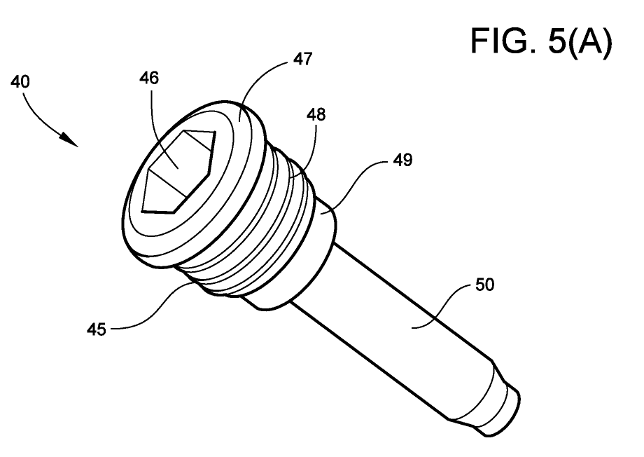

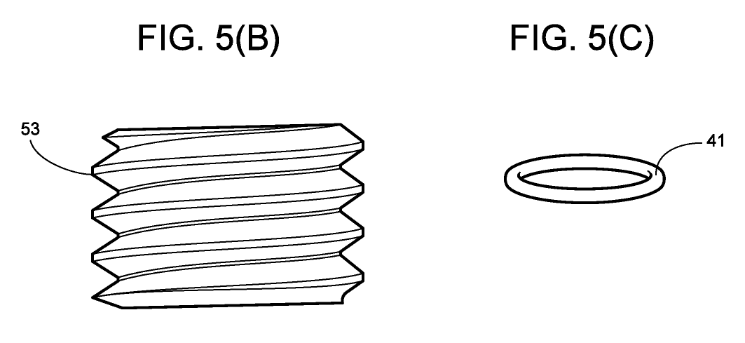

Optionally, a load-bearing component 41 (shown in FIG. 5(C)) such as a washer or other similar mechanism may be positioned in between the bulbous body 26 and the concave socket 12 to provide further controlled motion of the longitudinal member 20 with respect to the fixation component 10.

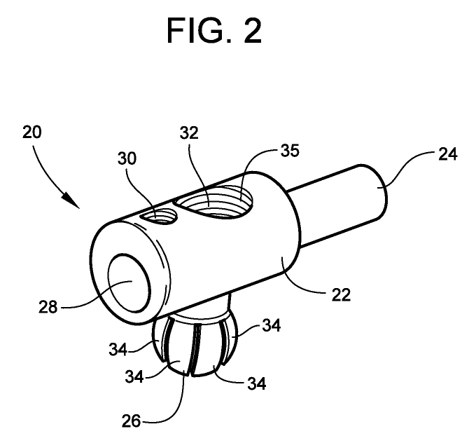

FIG. 2, with reference to FIG. 1, provides a front perspective view of longitudinal member 20. In FIG. 2, longitudinal member 20 includes a first body 22, an elongated body 24 and a bulbous body 26. Bored in first body 22 are a plurality of channels including elongation channel 28, optional setting channel 30, and securing channel 32. As shown, elongation channel 28 has a smooth bore while both setting channel 30 and securing channel 32 each have threads etched into an inner perimeter therein. Additionally, bulbous body 26 includes a plurality of slotted bendable flanges 34 that allow bulbous body 26 to expand when engaged within spherical female socket 12 of fixation component 10 at any allowable angle once the securing pin 40 is forced through. Since longitudinal member 20 is pivoting inside the female socket 12 of fixation component 10, screw assembly 1 is allowed to be inserted deeper into the bone without having the bone or anatomy prematurely limit the range of angulations of longitudinal member 20.

Elongation channel 28 is preferably configured as a substantially horizontal bore (i.e., with respect to the longitudinal axis of the first body 22 and elongated body 24) through the first body 22 and terminates at the securing channel 32. Setting channel 30 is a substantially vertical bore (i.e., with respect to the longitudinal axis of the first body 22 and elongated body 24) through the first body 22 and terminates at elongation channel 28. Furthermore, securing channel 32 is also a substantially vertical bore (i.e., with respect to the longitudinal axis of the first body 22 and elongated body 24), and is configured through first body 22 and bulbous body 26. Techniques for creating such bores as shown in FIG. 2 are well know to those skilled in the art.

Longitudinal member 20 also has threads 35 etched into first body 22, configured to mate with threads embedded in securing pin 40 (as described below). Setting channel 30 is similarly configured with threads etched into first body 22 and are configured to mate with threads embedded on a set screw 33 (shown in FIG. 5(B)).

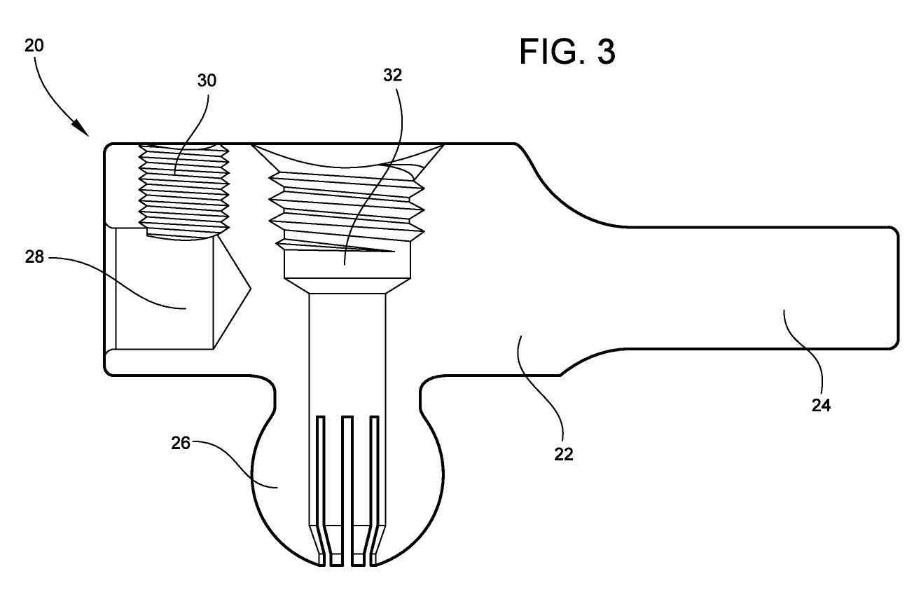

FIG. 3 illustrates a cross-sectional view of longitudinal member 20. As discussed above, FIG. 3 shows elongation channel 28 as a horizontal bore through first body, which terminates before intersecting with securing channel 28. Securing channel 28 is shown as a vertical bored through first body 22 and bulbous body 26. Moreover, setting channel 30 is shown as a vertical bore through first body 22 and terminating at elongation channel 28. Also shown in FIG. 3 are first body 22 and elongated body 24.

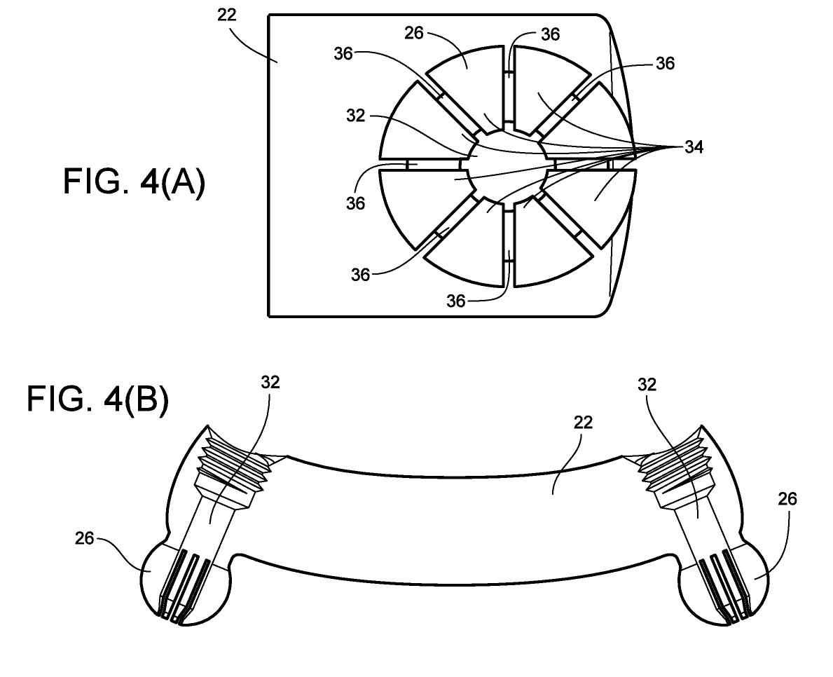

FIGS. 4(A) and 4(B), with reference to FIGS. 1 through 3, illustrate the longitudinal member 20 that includes bulbous body 26. As shown in FIGS. 4(A) and 4(B), longitudinal member 20 includes the expandable bulbous (or generally spherical) male body 26 for engaging the concave female socket 12 of fixation component 10. A plurality of axially spaced slots 36 are cut into bulbous body 26 forming a plurality of flanges 34, which expand once securing pin 40 is forced through securing channel 32 and cause the flanges 34 to outwardly project and expand. As a consequence, bulbous body 26 to expands into female spherical socket 12 of fixation component 10 at any allowable angle and thereby securing longitudinal member 20 to fixation component 10 via bulbous body 26.

FIG. 5(A), with reference to FIGS. 1 through 4(B), illustrates a side prospective view of securing pin 40. As shown, securing pin 40 includes an upper fastening portion 45 and a lower tip portion 50. Upper fastening portion 45 further includes fastening socket 46, pin head 47, threads 48, and connecting ring 49. As shown, fastening socket 46 is a hexagonal shape. Those skilled in the art would recognize that other configurations are possible—for example, fastening socket 46 may be square or any other polygonal shape or may be a linear slit or cross-slit in pin head 47. Threading 48 is embedded around an outer perimeter of upper fastening portion 45 and is configured to engage threads 35 etched into the inner perimeter of securing channel 32 of the longitudinal member 20. Connecting ring 49 is coupled to both the upper fastening portion 45 and lower tip portion 50. When upper fastening portion 45 and lower tip portion 50 are composed of different materials (as described in further detail below), connecting ring 49 provides additional strength in the coupling thereof.

Securing pin 40 may also comprise a multi-part assembly. For example, the upper fastening portion 45 of securing pin 40 may comprise titanium and the lower tip portion 50 of the securing pin 40 may comprise a ceramic material. Additionally, the lower tip portion 50 may comprise a mechanically harder material than the upper fastening portion 45. In such a configuration, longitudinal member 20 and the fixation component 10 may optionally comprise a first material, and the lower tip portion 50 of the pin 40 may comprise a material having a higher material hardness and compressive yield strength than the first material. Moreover, screw assembly 1 may further comprise a wear resistant ceramic coating (not shown) over longitudinal member 20 and the fixation component 10.

Assembly 1 can also be used as a dynamic rod system to complement artificial discs. According to this aspect of the embodiments herein, the outside of the bulbous body 26 and the inner spherical surface of female socket 12 are coated with a wear resistant ceramic coating. In this scenario, the securing pin 40 is not digging into the fixation component 10 and in fact is configured at a shorter length than some of the other embodiments. This allows some motion instead of rigid fixation and shares the load with the artificial disc disallowing excessive forces being applied to the artificial disc and increasing its functional life. For example, this occurs as a result of the ceramic coating, which may be used in the embodiments herein. As such, the bulbous body 26 of longitudinal member 20 and the female socket 12 of the bone screw 10 has a lower friction and higher wear resistance characteristics, thus improving the overall movement characteristics of the screw assembly 1.

FIG. 5(B), with reference to FIGS. 1 through 5(A), illustrates a set screw 53 used in accordance with the embodiments herein. Set screw 53 is dimensioned and configured to engage the setting channel 30 of the longitudinal member 20. The set screw 53 is used to secure a corresponding elongated body 24 of another longitudinal member 20, which connects inside elongated channel 28 to link two longitudinal members 20 together. Once the elongated body 24 of another longitudinal member 20 is positioned inside the elongation channel 28 and is in a desired position, the set screw 53 is inserted into the setting channel 30 such that the set screw 53 engages the elongated body 24 of another longitudinal member 20 and fixes it into a static position. In addition, while not shown in FIG. 5(B), set screw 53 also may include a fastening socket configured as a square or any other polygonal shape or may be a linear slit or cross-slit across a top of set screw 53.

FIG. 5(C) illustrates an optional load bearing component. Load-bearing component 41 includes, but is not limited to, a washer or other similar mechanism. Optionally, load bearing component 41 may be positioned in between the bulbous body 26 and the concave socket 12 to provide further controlled motion of the longitudinal member 20 with respect to the fixation component 10.

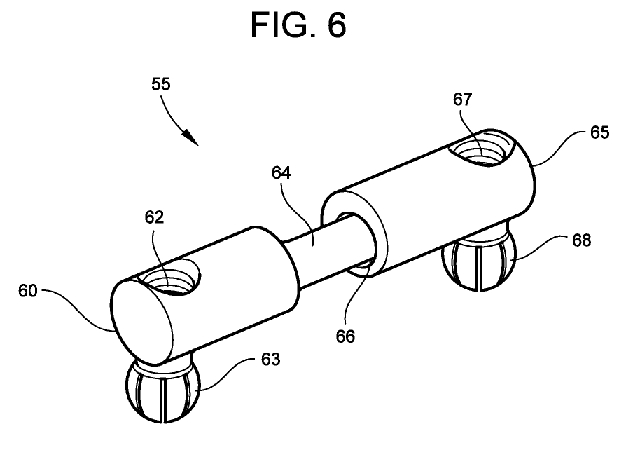

FIG. 6, with reference to FIGS. 1 through 5(C) illustrates a second embodiment herein, which shows a system 55 of two adjoining longitudinal members 60, 65 connected together. The elongation system includes male longitudinal member 60 and female longitudinal member 65. As shown, male longitudinal member 60 includes securing channel 62 positioned through the member 60 and through connected bulbous body 63, which then connects to a fixation component (i.e., similar to fixation component 10 illustrated in FIG. 1) via a securing pin (i.e. similar to the securing pin 40 illustrated in FIG. 5(A)). Female longitudinal member 65 includes a elongation channel 66 and securing channel 67 positioned through the member 65 and through connected bulbous body 68, which then connects to a fixation component (i.e., similar to fixation component 10 illustrated in FIG. 1) via a securing pin (i.e. similar to the securing pin 40 illustrated in FIG. 5(A)). Also shown in FIG. 6 is elongating member 64 of male longitudinal member 60 engaged with the elongation channel 66 of female longitudinal member 65. While not shown in FIG. 6, a corresponding setting channel and set screw may be incorporated with member 65 in a manner consistent with the first embodiment shown in FIGS. 1 through 5(B).

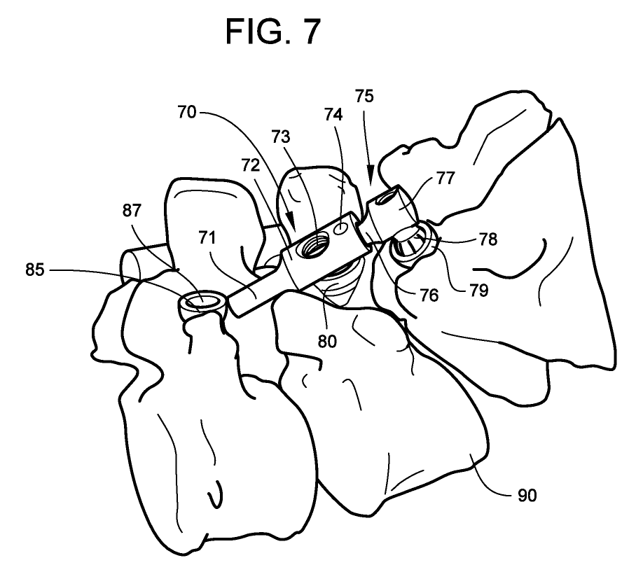

FIG. 7, with reference to FIGS. 1 through 6, illustrates a third embodiment herein showing two adjoining longitudinal members 70, 75 used for inter-connecting vertebral column 90. Similar to longitudinal member 20, shown in FIG. 2, longitudinal member 70 includes elongated body 71, first body 72, securing channel 73, and setting channel 74. While not shown in FIG. 7, longitudinal member 70 also includes an elongation channel and a bulbous body, where the bulbous body is securely seated in the female socket of fixation component 80. Longitudinal member 75 is an alternative embodiment to longitudinal member 70 and includes elongated body 76, first body 77, and bulbous body 78. Longitudinal member 75 also includes a securing channel to secure bulbous body 78 into the female socket of fixation component 79. Fixation components 79, 80, and 85 are shown as being secured in vertebral column 90 with female socket 87 visible.

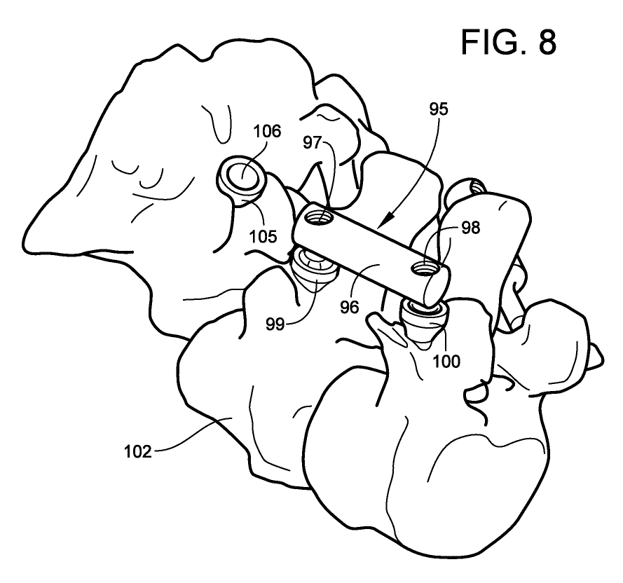

FIG. 8, with reference to FIGS. 1 through 7, illustrates a view of a fourth embodiment herein. As shown, longitudinal member 95 spans the length adjoining fixation component 99 and fixation component 100. Longitudinal member 95 includes first body 96, securing channel 97, and securing channel 98. While not shown in FIG. 8, longitudinal member 95 optionally includes an elongation channel, an elongated body, and a setting channel. Engagement of the bulbous end of the longitudinal member 95 with the fixation components 99, 100 is consistent with the embodiments shown in FIGS. 1 through 7 and described above. Accordingly, in FIG. 8 fixation component 105 comprises female socket 106, whereby fixation components 99 and 100 are similarly configured. Fixation components 99, 100, and 105 are each secured in vertebral column 102.

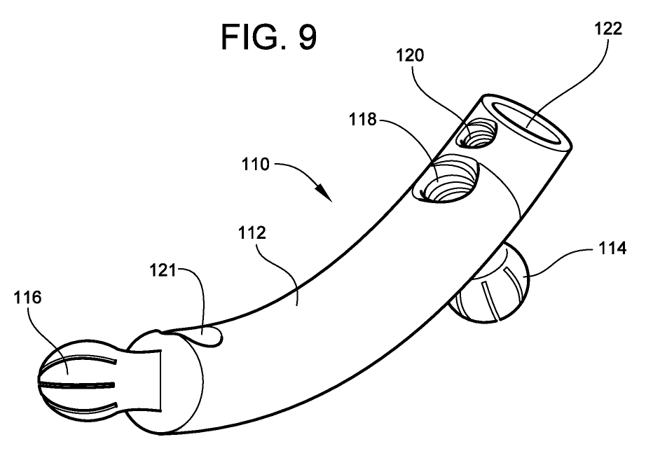

FIG. 9, with reference to FIGS. 1 through 8, illustrates a fifth embodiment herein. As shown, longitudinal member 110 includes a first body 112 and bulbous bodies 114, 116. First body 112 is slightly parabolic in shape and may, for example, be shaped to accommodate various curves present in human anatomy. Also shown in FIG. 9 are securing channel 118, optional setting channels 120, and elongation channel 122, which are configured in a manner consistent with the embodiments shown in FIGS. 1 through 8, and described above. In this embodiment, the bulbous body 116 is positioned substantially aligned along the curvilinear axis of the first body 112 or may be slightly angled with respect to the curvilinear axis of the first body 112 in order to engage a fixation component; for example, fixation component 105 in FIG. 8, that is not aligned with other fixation components 99, 100 and which may be positioned in a different plane compared with other fixation components 99, 100. As an example, in this embodiment, bulbous body 114 may engage fixation component 99 of FIG. 8, while bulbous body 116 may engage fixation component 105 in FIG. 8.

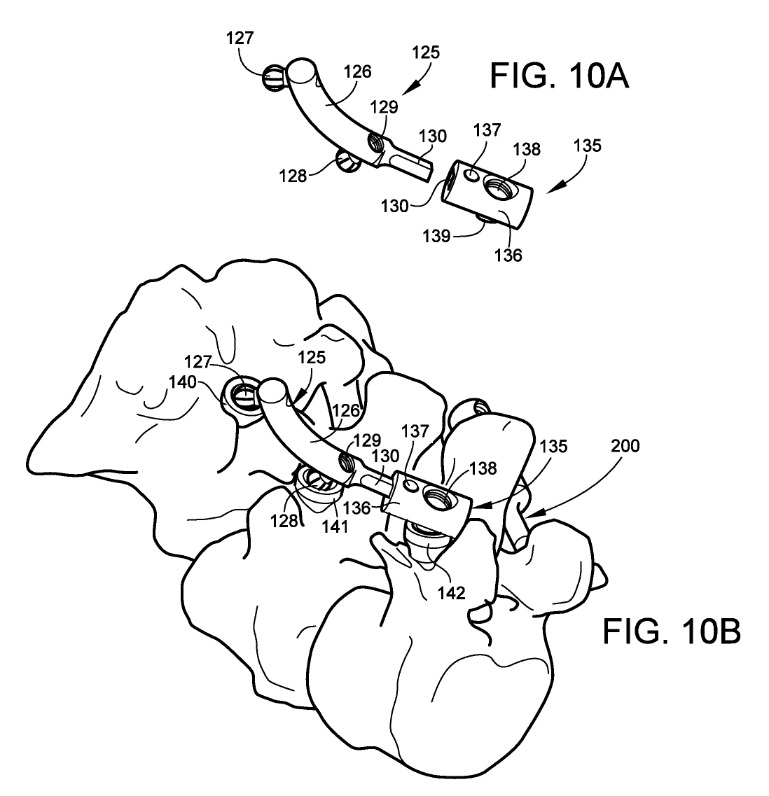

FIGS. 10(A) and 10(B), with reference to FIGS. 1 through 9, illustrate a sixth and seventh embodiment herein, respectively. FIG. 10(A) shows longitudinal member 125 with a first body 126, bulbous bodies 127 and 128, and elongated body 130. In addition, longitudinal member 125 includes securing channel 129. Similar to longitudinal member 110 in FIG. 9, longitudinal member 125 has a first body 126 with a slightly parabolic shape to allow bulbous bodies 127, 128 to engage fixation components positioned on different planes. Longitudinal member 135 includes first body 136, optional setting channel 137, and securing channel 138. Longitudinal member 135 also includes an elongation channel, which is shown in FIG. 10(A) as being “loaded” with elongated body 130 of longitudinal member 125.

In FIG. 10(B), longitudinal member 125 is fixedly coupled to fixation components 140, 141 via bulbous bodies 127, 128. To secure longitudinal member 125 to the fixation components 140, 141, a securing pin (not shown in FIG. 10, but shown in FIG. 1) is driven into a securing channel 129 to expand the bulbous body 127, 128 in the female sockets of the fixation components 140, 141, respectively. Longitudinal member 125 is coupled to longitudinal member 135 via elongated member 130, which is engaged with the elongation channel of longitudinal member 135. Elongated member 130 is fixed within the elongation channel via a set screw (such as set screw 53) driven into setting channel 137. Longitudinal member 135 is fixedly coupled to fixation component 142. Similar to longitudinal member 125, securing channel 138 has a securing pin (not shown) driven through first body 136 to expand a bulbous body 139 in the female socket of fixation component 142. Also shown in FIG. 10(B) is another assembly 200, which may be configured on adjacent lamina.

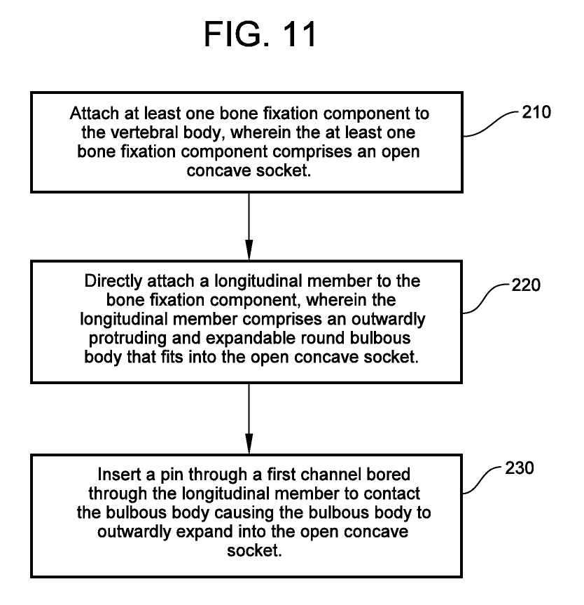

FIG. 11, with reference to FIGS. 1 through 10(B), is a flow diagram illustrating a method of engaging a pedicle fixation assembly (for example, assembly 1) to a vertebral body (for example, vertebral body 102) according to an embodiment herein. The method comprises attaching (210) at least one bone fixation component 10 to the vertebral body 102, wherein the at least one bone fixation component 10 comprises an open concave socket 12. The next step involves attaching (220) a longitudinal member 20 to the bone fixation component 10, wherein the longitudinal member 20 comprises an outwardly protruding and expandable round bulbous body 26 that fits into the open concave socket 12. Thereafter, a pin 40 is inserted (230) through a first channel 32 bored through the longitudinal member 20 to contact the bulbous body 26 causing the bulbous body 26 to outwardly expand into the concave socket 12.

The method described in FIG. 11 may also be performed by an automatic apparatus, or an otherwise non-human device, or encoded within a computer-readable medium. Automatic devices may include, for example, a robotic arm or remote controlled automata. In general, such devices may assist a human operator or be fully automated (i.e., without the aid of human input). Example of the former include surgical procedures performed via a remote control and devices used in telemedicine, while examples of the latter include a robotic surgeon and nursing robots, which are fully automated but assist a human surgeon.

The embodiments herein provide a headless polyaxial pedicle screw assembly 1, or generally a posterolateral fixation system, which may be used anteriorly or posteriorly, and which is capable of being utilized in surgeries to achieve anterior lumbar interbody fusion, posterior lumbar interbody fusion, transverse lumbar interbody fusion, correct degenerative disc disease, adult and pediatric scoliosis as a fixation device, and posterior cervical fusion. The embodiments herein are improvements in the field of surgical lumbar and thoracic spine treatment. An adaptation of the embodiments herein may also be utilized in cases where it is not pertinent to perform a spinal fusion; the only requirement being motion limitation in the particular motion segment.

Moreover, the embodiments herein provide a polyaxial spinal screw assembly 1 that can become rigid similar to a monoaxial screw inter-operatively on demand. The embodiments herein also offer the surgeon more lateral range of motion than conventional products by utilizing the space under the longitudinal member 20 to provide a bigger arc of rotation. The embodiments herein also allow for polyaxial direct connection from the longitudinal member 20 to the bone anchor 10. Furthermore, by reducing the amount of components, and therefore the amount of foreign materials to be implanted during the surgical procedure, the embodiments herein provide a patient with an improved prognosis for better and faster rehabilitation.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. An assembly comprising:
a longitudinal member comprising an outwardly protruding and expandable round bulbous body;
a fixation component directly connected to said bulbous body, wherein said fixation component receives said bulbous body; and
a pin engaged within said longitudinal member via a first channel bored through said longitudinal member and contacting said bulbous body causing said bulbous body to outwardly expand.

2. The assembly of claim 1, wherein said fixation component comprises a concave socket that receives said bulbous body of said longitudinal member.

3. The assembly of claim 2, wherein said longitudinal member comprises a substantially planar lower surface, wherein said bulbous body extends from said lower surface of said longitudinal member, and wherein said concave socket cups said expandable bulbous body.

4. The assembly of claim 1, wherein said fixation component comprises a bone screw.

5. The assembly of claim 1, wherein said fixation component comprises a hook.

6. The assembly of claim 1, further comprising a load-bearing mechanism positioned between said bulbous body and an upper portion of said fixation component.

7. The assembly of claim 1, further comprising a set screw engaged with said longitudinal member via a second channel bored through said longitudinal member.

8. The assembly of claim 1, wherein said pin engages said fixation component.

9. The assembly of claim 7, wherein each of said first channel and second channel is etched with threads.

10. The assembly of claim 1, wherein said bulbous body of said longitudinal member comprises a plurality of slots separating a plurality of bendable flanges of said bulbous body.

11. A system comprising a first assembly and a second assembly, wherein said first assembly comprises:
a first longitudinal member comprising:
a first elongated body outwardly extending from at least one longitudinal end of said first longitudinal member; and
an outwardly protruding and expandable round bulbous body extending from a lower portion of said first longitudinal member, wherein said lower portion of said first longitudinal member is positioned substantially transverse to said at least one longitudinal end of said first longitudinal member;
a fixation component directly connected to said bulbous body, wherein said fixation component receives said bulbous body; and
a pin engaged within said longitudinal member via a first channel bored through said first longitudinal member and contacting said bulbous body causing said bulbous body to outwardly expand.

12. The system of claim 11, wherein said first longitudinal member further comprises a first elongation channel bored therein, and wherein said second assembly comprises a second longitudinal member comprising:
a second elongated body outwardly extending from at least one longitudinal end of said second longitudinal member; and
a second longitudinal member comprising a second elongation channel bored therein.

13. The system of claim 12, wherein said first elongation channel accepts said second elongated body, and wherein said second elongation channel accepts said first elongated body.

14. The system of claim 13, wherein any of said first longitudinal member of said first assembly and said second longitudinal member of said second assembly further comprises a hole that receives a set screw.

15. The system of claim 14, wherein said set screw of said first assembly engages said second elongated body.

16. The system of claim 14, wherein said set screw of said second assembly engages said first elongated body.

17. An assembly comprising:
a longitudinal member comprising at least two outwardly protruding and expandable round bulbous bodies;
at least two fixation components directly connected to said bulbous bodies; and
at least two pins engaged within said longitudinal member via channels bored through said longitudinal member, wherein each pin is set within each channel and contacts a bulbous body causing said bulbous body to outwardly expand to engage a fixation component.

18. The assembly of claim 17, wherein each of said bulbous bodies are angled with respect to each other.

19. The assembly of claim 17, wherein said longitudinal member comprises at least one of an elongated body and an elongation channel.

20. A method of engaging a pedicle fixation assembly to a vertebral body, said method comprising:
attaching at least one bone fixation component to said vertebral body, wherein said at least one bone fixation component comprises an open concave socket;
directly attaching a longitudinal member to said bone fixation component, wherein said longitudinal member comprises an outwardly protruding and expandable round bulbous body that fits into said open concave socket; and
inserting a pin through a first channel bored through said longitudinal member to contact said bulbous body causing said bulbous body to outwardly expand into said open concave socket.
